(19) United States
(12) Patent Application Publication
(10) Pub. No.: US 2008/0132958 A1
(43) Pub. Date: Jun. 5, 2008

(54) ORTHOPEDIC COMPRESSION SCREW

(75) Inventors: Uwe Pech, Tuttlingen (DE); Roland Worner, Tuttlingen-Mohringen (DE); Michael Vitek, Wien (AT)

Correspondence Address: BACHMAN & LAPOINTE, P.C., 900 CHAPEL STREET, SUITE 1201, NEW HAVEN, CT, 06510, US

(73) Assignee: NORMED MEDIZIN-TECHNIK VERTRIEBS GMBH, Tuttlingen (DE)

(21) Appl. No.: 11/947,845

(22) Filed: Nov. 30, 2007

(30) Foreign Application Priority Data
Nov. 30, 2006 (DE) 10 2006 056 950.4

(51) Int. Cl. A61B 17/56

(52) U.S. Cl. 606/305; 606/301

(57) ABSTRACT

The invention relates to an orthopedic compression screw, in particular a compression screw for osteosynthesis and/or fixation of bone segments (12, 14), with a threaded portion (20) and a screw head (18), the screw head (18) widening conically toward its distal end and the screw being provided along its longitudinal axis (A) with a bore (26) for interaction with an orthopedic guide element, in particular a guide wire, and the periphery of the screw head (18) having at least one flat that forms a plane outer surface (30) preferably extending parallel to the longitudinal axis.

### BACKGROUND OF THE INVENTION

The present invention relates to an orthopedic compression screw according to the preamble of the main claim. Devices of this kind are generally known from the prior art and are used particularly in orthopedic procedures carried out on the feet or hands. Screws of this kind, usually subjected to a tensile load when inserted, are used to fix bone parts to one another (again) in a desired connection position, so as to once more ensure the mechanical stability of the particular bone, which has been separated by a fracture or by a deliberate surgical measure.

Conventional techniques present problems particularly when applied to surgery of so-called flexible hammer toes and in view of the resulting fixation requirements. Deliberate shortening of the metatarsal bones in the metaphyseal and diaphyseal areas also represents an orthopedic challenge where, in addition to the problem of fixation by means of suitable screws, a further particular challenge lies in creating means with which a predetermined shortening of the bone can be permitted by predetermined bone cuts and removal in particular of disk-shaped and wedge-shaped bone segments.

The object of the present invention is therefore to make available an orthopedic compression screw which is suitable in particular for operations in surgery of the hands and feet and which permits the flexible, in particular substantially inclined insertion for bone fixation or osteosynthesis simply and with minimal surgical outlay and at the same time takes up considerable holding forces and, in respect of the tissue surrounding the operating site, is only very slightly invasive.

The object is achieved by the orthopedic compression screw with the features described herein and by the system for extraction of a bone segment also described herein, advantageous developments of the invention also being set forth below.

### SUMMARY OF THE INVENTION

In an advantageous manner according to the invention, the periphery of the (partially) conically widened screw head of the orthopedic compression screw according to the invention is provided at least with one flat, preferably with two or more flats, such that, with minimal bone loss, the screws provided with a small head can be introduced very flat or obliquely (for example relative to a longitudinal axis of the bone), without the head disturbing the soft tissue parts at the operating site. Rather, the configuration claimed according to the invention allows the screw head to be embedded, such that the screws according to the invention are suitable especially for operating sites where there is a minimal covering of the bone by soft tissue. At the same time, the screw according to the invention allows the tensile force to be effectively introduced into or onto the bone via the cone shape of the head.

It is particularly preferable to define the geometry of the screw head (including the maximum distal diameter of the screw head, the angle inclination of the conical screw head jacket relative to the longitudinal axis of the screw, the groove depth of the thread or the diameter of the shaft portion, and the distance between the outer surfaces advantageously formed according to one development in a pair parallel to each other) in relative positions to one another, as in the dependent claims. In this way, it is possible to achieve in a particularly expedient manner the intended application purpose of osteosynthesis or bone fixation on sensitive bones of the extremities, in particular the bones of the feet, such that mechanical stability is advantageously balanced with ease of implantation and operation, a secure hold in the bone and optimized bone loss through insertion of the screw.

The screw is preferably realized as a titanium element or a titanium-containing or titanium-coated element, and follows the Torx principle for the purpose of optimized introduction of the force by the surgeon implanting the screw.

Independent protection in the context of the invention is claimed for a system for extracting a preferably wedge-shaped or disk-shaped bone segment from a bone, said system comprising an abutment or a guide surface for a cutting (sawing) procedure in the bone, and an orthopedic compression screw, in particular the compression screw according to the main claim, then permitting reconnection of the bone after extraction of the bone segment. According to the invention, the jig allows the cutting procedure for the extraction to be carried out in a constructively simple and surgically elegant and favorable manner, such that, according to the invention, the fixability of the jig on a guide element, in particular a guide wire (typically used: so-called Kirschner wire), is simplified and, in addition, the fixed angle adjustability of the jig designed, according to one development, as a modular multi-part assembly makes it particularly easy, at the operating site, to provide and orient the for example wedge-shaped extraction cut. In addition or alternatively, provision is advantageously made for the jig to be scissor-shaped and for the angles to be adjusted variably (preferably using a scale).

In addition, in the context of the present invention and disclosure, protection is claimed for an implanting, sawing, working and operating method, as set forth in the present documents, including the attached drawings of the illustrative embodiments.

As a result, the technical medical problems described at the outset are solved by the present devices in a surprisingly simple and elegant manner; it is expected that, by virtue of the orthopedic compression screw according to the invention and its special geometry and by virtue of the extraction and fixation system according to the invention, the difficult area of foot (and hand) surgery may be enriched by fundamentally novel and advantageous technology.

### BRIEF DESCRIPTION OF THE DRAWINGS

Other advantages, features and details of the invention will become clear from the following description of preferred illustrative embodiments and from the drawings, in which:

FIG. 1 is a schematic representation illustrating the area in which the orthopedic compression screw according to the invention is used when implanted for fixation of two bone parts of a bone of the foot;

FIG. 2 shows a longitudinal section through the screw from FIG. 1 according to a first preferred embodiment;

FIG. 3 shows an end view of the screw from FIG. 2;

FIG. 4 shows a detail of the screw thread as indicated by “X” in FIG. 2;

FIG. 5 shows a perspective view of the jig that can be used in the system according to the invention and that is of a modular configuration in the illustrative embodiment;

FIG. 6 shows a view of the jig from FIG. 5, with a second jig element which presents a different abutment angle compared to the abutment angle of the jig element from FIG. 5 (for generating a wedge-shaped bone segment by means of two cuts made at an angle to each other);

FIG. 7 is a schematic representation illustrating the use of the jig arrangement from FIGS. 5 and 6;

FIG. 8 shows a perspective view of an alternative jig, and

FIG. 9 is a view showing the possible uses of the jig from FIG. 8.

### DETAILED DESCRIPTION

The geometries and geometrical relationships disclosed in the illustrative embodiments to be described below apply particularly as disclosed in relation to the invention, likewise the recognizable method steps.

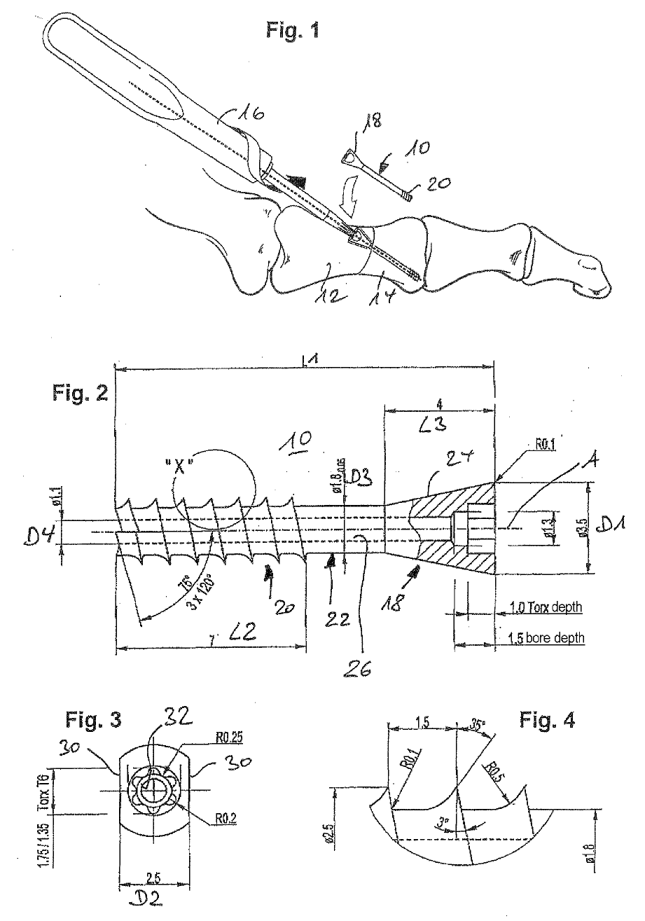

Thus, FIG. 1 is a schematic representation showing how a compression screw 10 according to a first embodiment of the present invention is inserted (by means of a screwdriver 16 shown only by way of example) obliquely into a bone that has been divided into two halves 12, 14, such that a screw head 18 of the screw 10 can be inserted into the first bone part 12 in a manner substantially inclined relative to a longitudinal axis of the bone 12, 14, in such a way that the head 18 disappears completely in the bone substance, while a threaded portion 20 of the screw 10 sits in the adjacent bone part 14, with the result that the parts 12, 14 can be fixed onto each other by the action of the screw.

In the illustrative embodiment shown, not only does the detailed geometry of the screw 10, as seen from FIGS. 2, 3 and 4, mean that the screw can be inserted obliquely and be fully embedded, as is indicated in FIG. 1, the depicted geometry also ensures that the head 18 and thread 20 can have the best possible mechanical fit, associated with minimized loss of bone in the area of the head 18. Thus, the screw 10, which can typically be configured in different lengths according to a respective intended use, has a total length L1, as shown in FIG. 2, with the threaded portion extending along a subsidiary length L2, and the screw head extending in the axial direction along a wedge length L3. Between the threaded portion 20 and the screw head 18 in the illustrative embodiment shown, an unthreaded shank portion 22 is formed, the lengthwise extent of which is obtained from the difference L1−L2−L3.

Perpendicular to the longitudinal axis or axis of symmetry A through the screw in FIGS. 2 and 3, there is in the first instance the diameter D1 (more precisely the maximum distal diameter) of the screw head 18, which describes the maximum diameter and entails a conical widening 24 of the screw head 18 (inclined, in the illustrative embodiment, by an angle of 11° relative to the longitudinal axis A); the widening is advantageously provided such that the maximum distal diameter D1 of the screw head relative to the diameter D3 of the unthreaded shank portion 22 (corresponding, in the illustrative embodiment, to the diameter at the thread root of the threaded portion 22) is typically ca. 2:1, from which it follows that D3 is typically approximately 45 to 55% of D1 preferably. According to the illustrative embodiment, provision is also advantageously made that the axial length L3 of the screw head 18 can be from 1 to 1.3 times ( 9/7) D1, and that, alternatively or in addition, a diameter D4 of an axial longitudinal bore 26 formed for interaction with orthopedic guide wires (in particular a Kirschner wire) is approximately 55 to 65% of the diameter D3 or approximately 30 to 35% of the diameter D1, in all the present geometries that can be seen from FIGS. 2 to 4 the numbers given are in millimeters, the concretely specified distances, diameters and proportions apply as disclosed, as also do the ratio ranges cited in the present description of the invention and illustrative embodiment.

It will be clear in particular from the end view in FIG. 3 that the screw head 18 is flattened on both sides, in such a way that two flat faces 30 are formed parallel to each other and on each side of the longitudinal axis A and are at a distance D2 from each other. This distance D2 is preferably approximately 4/7 to 6/7 of the maximum distal diameter D1 of the screw head. As has been explained at the outset, this geometry permits the desired optimized effect of the screw head in respect of retaining force, bone consumption and implantation. The end view in FIG. 3 also illustrates the Torx screw attachment 32.

Further to the view in FIG. 4, and as an enlarged view of the detail “X” from FIG. 2, FIG. 4 also illustrates the special cross-sectional thread configuration of the threaded portion 18 in the illustrative embodiment shown, particularly in respect of the angle and radius geometries.

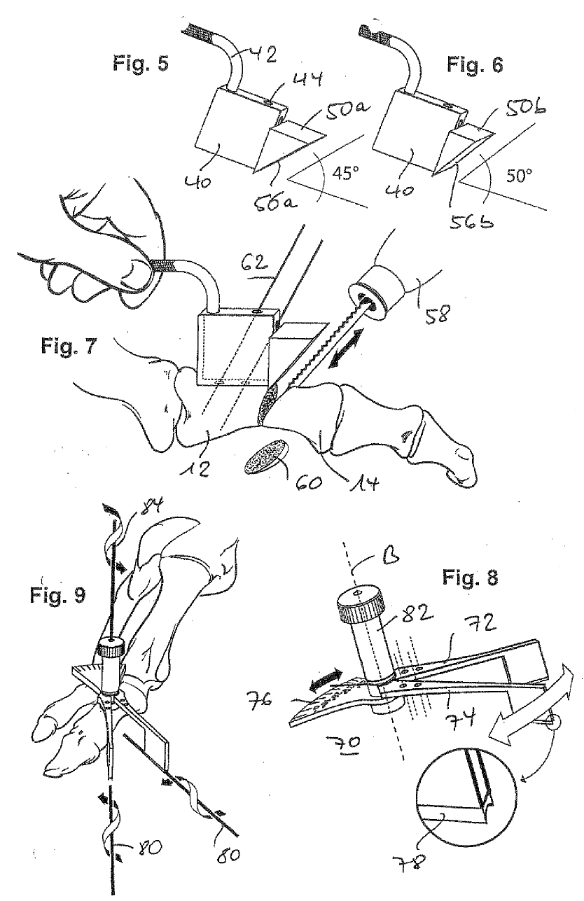

FIGS. 5 to 7 illustrate how, according to a further preferred illustrative embodiment of the invention, the jig can be provided with a structurally simple and effective design. It consists of a body (main element 40), which has an attached grip portion 42, is provided with apertures 44 for inserting and guiding Kirschner wires, and cooperates with a second, wedge-shaped jig element 50. More precisely, this second jig element 50, as shown in FIGS. 5 and 6, is supplied as an exchangeable pair of wedge elements, and these wedge elements, the variants 50a and 50b are shown, each bear on an end face of the body 40 and present an abutment surface 56 (shown as variants 56a and 56b for the angles 45° and 50° in FIGS. 5 and 6) along which, as can be seen from the schematic representation in FIG. 7, a sawing device 58 is then guided and, in two consecutive cuts with the elements 56a and 56b in place, can extract a wedge-shaped bone segment 60. FIG. 7 additionally illustrates the guide wires (Kirschner wires 62) engaging in the guides 44. An approximation or closing together of the illustrated bone sections 12, 16, after the bone segment 60 has been extracted, then leads to the geometric configuration according to FIG. 1, such that the screw 10 can then be inserted for fixing the bone parts 12, 16.

FIG. 8 illustrates an alternative jig with a variably adjustable angle. The scissor-like jig element 70 shown in FIG. 8 comprises a pair of branches 72, 74, which between them enclose an angle indicated by a scale 76. As is shown in FIG. 9, guide wires 80 can then be inserted along the branches 72, 74, which have a groove 78 on the underside, and a further guide wire 84 can also be inserted along a longitudinal pivot axis B of a pivot hinge unit 82 that pivotably connects the branches 72, 74.

1. Orthopedic compression screw, in particular a compression screw for osteosynthesis and/or fixation of bone segments (12, 14), with a threaded portion (20) and a screw head (18), the screw head widening conically toward its distal end and the screw being provided along its longitudinal axis (A) with a bore (26) for interaction with an orthopedic guide element, in particular a guide wire, characterized in that the periphery of the screw head (18) has at least one flat that forms a plane outer surface (30).

2. Screw according to claim 1, wherein the plane outer surface (30) extends parallel to the longitudinal axis.

3. Screw according to claim 1, characterized in that the flat has a pair of outer surfaces (30) that lie opposite each other, relative to the longitudinal axis, and are parallel to each other and to the longitudinal axis.

4. Screw according to claim 3, characterized in that the distance (D2) between the outer surfaces (30) is between 4/7 and 6/7 of the maximum distal diameter (D1) of the screw head.

5. Screw according to claim 1, characterized in that an axial length (L1) of the conical screw head is 9/7 to 7/7 of the maximum distal diameter (D1) of the screw head.

6. Screw according to claim 1, characterized by an unthreaded shank portion (22) provided between the threaded portion and the screw head.

7. Screw according to claim 6, characterized in that the diameter (D3) of the unthreaded shank portion and/or a diameter of the threaded portion at the thread root is 40 to 60% of the maximum distal diameter (D1) of the screw head.

8. Screw according to claim 6, wherein the diameter (D3) of the unthreaded shank portion and/or a diameter of the threaded portion at the thread root is 45 to 55% of the maximum distal diameter (D1) of the screw head.

9. Screw according to claim 1, characterized in that a diameter (D4) of the bore is between 60% and 70% of the diameter (D3) of the unthreaded shank portion and/or between 30% and 35% of the maximum distal diameter of the screw head.

10. Screw according to claim 1, characterized in that an axial length of the screw head (L3) is 10% to 30% of the screw length (L1).

11. Screw according to claim 1, characterized in that a cone angle of the screw head relative to the longitudinal axis is 100 to 150.

12. Screw according to claim 1, characterized in that a cone angle of the screw head relative to the longitudinal axis is 110 to 130.

13. Screw according to claim 1, characterized in that the screw is a Torx screw.

14. System for extracting a preferably wedge-shaped or disk-shaped bone segment (60) from a bone of the hand or foot and for subsequent fixation of the bone (12, 14), comprising a jig that presents an abutment and/or a guide surface (56a, 56b) for a bone-sawing device (58) and is to be placed on the bone, and comprising an orthopedic compression screw designed to fix the bone at the extraction position, wherein the orthopedic compression screw comprises a screw according to claim 1.

15. System according to claim 14, characterized in that the jig is designed for interaction with a guide element (62), in particular an orthopedic guide wire, that can be fixed in bone.

16. System according to claim 14, characterized in that, when the jig is placed on the bone, the abutment or the guide surface forms a fixed angle with respect to the line perpendicular to the longitudinal axis of the bone, the fixed angle being between 40 and 60°.

17. System according to claim 14, characterized in that, when the jig is placed on the bone, the abutment or the guide surface forms a fixed angle with respect to the line perpendicular to the longitudinal axis of the bone, the fixed angle being between 45 to 55°.

18. System according to claims 14, characterized in that the jig has a multi-part design and comprises a first jig element (40), with securing means (44) for the bone, and a preferably wedge-shaped second jig element (50a, 50b) which can be placed onto the first jig element and presents the abutment or guide surface.

19. System according to claim 18, characterized by a plurality of second jig elements (50a, 50b) which are differently graded in terms of their respective fixed angles of the abutment or of the guide surface.

20. System according to claim 14, characterized in that the jig (70) has a scissor-like design for the adjustment, preferably by means of a scale (76), of a sawing angle and/or abutment angle for the bone-sawing device.
